(19) United States
(12) Patent Application Publication
(10) Pub. No.: US 2020/0018749 A1
(43) Pub. Date: Jan. 16, 2020

(54) PLUG-IN EXPERTISE FOR PATHOGEN IDENTIFICATION USING MODULAR NEURAL NETWORKS

(71) Applicant: INDEVR, INC., Boulder, CO (US)

(72) Inventors: Andrew W. SMOLAK, Boulder, CO (US); Robert STOUGHTON, Boulder, CO (US); Amber W. TAYLOR, Boulder, CO (US)

(21) Appl. No.: 16/470,954

(22) PCT Filed: Dec. 20, 2017
(86) PCT No.: PCT/US17/67651

§ 371 Date: Jun. 18, 2019

Related U.S. Application Data
(60) Provisional application No. 62/436,934, filed on Dec. 20, 2016.

(51) Int. Cl. G01N 33/554; C12Q 1/04; G01N 33/567; G01N 33/569; G01N 33/68; G16B 20/00; G16B 40/20; G16B 40/30; C12Q 1/70

(52) U.S. Cl. CPC G01N 33/554; C12Q 1/04; G01N 33/567; G01N 33/569; G16B 25/00; G16B 20/00; G16B 40/20; G16B 40/30; C12Q 1/70; G01N 33/6893

(57) ABSTRACT

Provided herein are methods for characterizing pathogens based on data profiles generated by an analyzer. The provided methods allow for rapid identification and characterization of emergent pathogens or mutations by allowing for facile updates to the established pathogen data used by learning algorithms, while not altering the independent learning algorithms themselves.

### CROSS-REFERENCE TO RELATED APPLICATIONS

This application claims the benefit of priority from U.S. Provisional Patent Application No. 62/436,934, filed Dec. 20, 2016, which is incorporated herein by reference in its entirety.

### STATEMENT REGARDING FEDERALLY SPONSORED RESEARCH OR DEVELOPMENT

This invention was made with government support under Contract number HHSO100201400010C awarded by the Biomedical Advanced Research and Development Authority (BARDA), Office of the Assistant Secretary for Preparedness and Response, U.S. Department of Health and Human Services. The government has certain rights in the invention.

### BACKGROUND OF INVENTION

Modern clinical practice often relies on typing or genotyping to effectively diagnose and treat pathogenic infection. In response to this need, a range of diagnostic approaches have been developed providing clinically relevant information. Biomarker identification approaching, including RT-PCR based probe sequence amplification or immunoassays are desirable because they provide rapid sample evaluation. One promising example is microarray based methods for pathogen identification. Advantages of microarray techniques include the potential for greater diagnostic information content given the use of multiple, complementary capture sequences. These techniques also provide for rapid and sensitive optical readout and are compatible with straightforward sample processing and handling, thus providing the potential for point of care applicability.

In the context of influenza treatment, for example, micro-array based assays have emerged as a particularly promising platform for providing accurate and rapid characterization of influenza type, subtype, and seasonal strain information [see, e.g., Heil, G L. et al. “MChip, a low density microarray, differentiates among seasonal human H1N1, classical swine H1N1, and the 2009 pandemic H1N1”, Influenza Other Respir Viruses 2010, 4(6), 411-416, Moore, C L et al., “Evaluation of MChip with Historic A/H1N1 Influenza Viruses Including the 1918 “Spanish Flu'” J Clin Microbiol 2007, 45(11), 3807-3810; and U.S. Patent Publications 2009/0124512 and 2010/0130378].

Despite these advantages, microarray techniques suffer from limitations in comparison to more comprehensive analysis such as full genomic sequencing. Particularly, microarrays have challenges associated with identifying emergent pathogen strains or mutations. Because new microarray techniques rely on pattern recognition to identify and characterize pathogens, emergent strains or mutations cannot be identified using microarray techniques until an associated pattern is identified. Furthermore, new strains or mutations may be genetically similar to known strains and result in a similar microarray pattern and resulting analysis may mischaracterize an emergent strain or mutation as an existing strain, thereby failing to recognize that the pathogen is different than known strains.

The identification of emergent strains is further complicated by regulatory requirements, such as those required by the Food and Drug Administration (FDA). For example, in software-based analysis of microarrays, the FDA may require a testing and approval process for the module of software which identifies or characterizes a particular strain or aspect of a strain. Thus, updating modules or introducing new modules corresponding to emergent or mutated strains may require FDA testing and approval, delaying the ability of technicians to modify microarray techniques in order to identify new pathogen or pathogen characteristics, including in cases of emergent pandemics.

It will be appreciated from the foregoing that there is currently a need in the art for improved systems and methods of pathogen identification, typing and subtyping. In particular, systems and methods of providing reliable identification of emergent pathogen strains or characteristics which may provide rapid analysis of pathogens, including identifying or collecting data regarding mutated or new pathogen strains.

### SUMMARY OF THE INVENTION

Provided herein are microarray-based systems and methods for pathogen identification and characterization. Aspects of the invention implement run-time interpreted definition files and compiled supervised learning algorithms for data analysis to enhance the flexibility of pathogen detection devices and software. Embodiments of the invention, for example, utilize structured logical combinations of the output of independent learning algorithms relying on data from both known (previously identified and analyzed) and emergent (new strains or mutations) to provide an efficient and rapid pathway to clinically and epidemiologically relevant diagnostic information. The independent learning algorithms may include artificial neural networks.

Pathogen analysis methods may implement machine learning using training data sets corresponding to both well-characterized samples having known properties and data sets corresponding to emergent or previously unknown samples to provide pathogen characterization. Pathogen characterization includes pathogen type, subtype, whether a pathogen is an emergent strain or has a mutation, is a seasonal strain, and whether a pathogen has specific markers. The structured supervised learning aspect of some embodiments is compatible with straightforward retraining of supervised learning algorithms to respond to mutations due to antigenic drift or antigenic shift and characterize new pathogen strains.

In an aspect, provided is a method for characterizing a target pathogen comprising: (i) providing a sample derived from material potentially containing the target pathogen to a sample analyzer; (ii) generating a profile corresponding to the sample from the sample analyzer; (iii) analyzing the profile using a plurality of independent learning algorithms and one or more emergent pathogen parameter definition files, wherein: each of the independent learning algorithms is taught and compiled using a profile corresponding to one or more known pathogens; at least a portion of the independent learning algorithms independently provide: (a) a known pathogen parameter output of the target pathogen based on the one or more known pathogen parameters; and (b) an emergent pathogen parameter output of the target pathogen based on the one or more emergent pathogen parameter definition files, wherein the emergent pathogen parameter output occurs without recompiling any of the learning algorithms; wherein the emergent pathogen definition files allow the independent learning algorithms to provide the emergent pathogen parameter output without recompiling the learning algorithms or altering the known pathogen parameter outputs; and (iv) combining the known pathogen parameter outputs and emergent pathogen parameter outputs to make a pathogen determination, thereby characterizing the one or more target pathogens.

The provided methods are versatile and may be utilized with a variety of profiles or forms of data analysis, including microarrays and their corresponding intensity profiles. Additionally, the provided methods may determine different pathogen characteristics including whether the characterized pathogen cannot be characterized as a known strain and is an emergent strain or mutated strain. The provided methods can further characterize or identify emergent strains by utilizing updates which are separate from the core analysis by entering an open mode, in which additional emergent data is utilized in the pathogen characterization.

In embodiments, for example, the profile is an intensity profile map, a mass spectroscopy spectrum, an amino acid sequence or a nucleotide sequence. In an embodiment, the profile is an intensity profile map obtained from a microarray having a plurality of capture sequences, each capture sequence configured to bind to a target sequence of interest.

In an embodiment, the characterization is an identification of the presence or absence of the target pathogen. In embodiments, the characterization is an identification of one or more pathogen parameters of the target pathogen indicative of an emergent pathogen, corresponding to an unknown or null value of one or more of the known pathogen parameters. In some embodiments, for example, the pathogen parameters are selected from the group consisting of: type, subtype, genotype, absence of pathogen, strain, lineage, seasonality, mutation presence or absence, marker presence or absence, virulence, novelty, species of origin and any combination thereof.

In embodiments, each of the independent learning algorithms is independently trained to evaluate a single pathogen parameter of a target pathogen. In embodiments, at least a portion of the independent learning algorithms are independent artificial neural network (ANN) algorithms. In embodiments, the independent learning algorithms are supervised learning algorithms, for example, a support vector machine; a decision tree; a clustering algorithm, a Bayesian network, a random forest, a logistic regression algorithm, a K-nearest neighbor algorithm, and any combination thereof. In an embodiment, the target pathogen is one or more influenza viruses.

In an embodiment, the known pathogen parameters correspond to one or more of influenza A, influenza B, influenza A seasonal H1N1 viral strains, influenza A seasonal H3N2 viral strains, or influenza A non-seasonal viral strains. In some embodiments, the known pathogen parameters correspond to one or more of: H1, H2, H3, H5, H7 and H9 hemagglutinin subtypes and N1, N2, N7, N8 and N9 neuraminidase subtypes. In embodiments, the target pathogen is influenza A and at least one of the plurality of independent learning algorithms provides outputs corresponding to HA subtype and at least one of the plurality of independent learning algorithms provides outputs corresponding to NA subtype.

In some embodiments, the sample is a material potentially containing the pathogen is a biological material from a human or a non-human animal, an isolate or a culture. The biological material may contain one or more influenza viruses.

In some embodiments, the sample analyzer is a microrarray, a genetic sequencer, a protein sequencer or a mass spectrometer. In an embodiment, for example, the sample analyzer is a microarray.

The provided methods include methods for updating definition files allowing for characterization of new or emergent pathogens without altering the compiled (often approved by regulators) algorithms. This potentially allows for the characterization of new pathogens without new software versions or updates, which may require regulatory oversight. Additionally, it increases the speed at which new strains or mutations may be properly diagnosed by laboratories.

In embodiments, the emergent pathogen parameter definition file is periodically provided to the sample analyzer. In an embodiment, for example, the emergent pathogen parameter definition file corresponds to a newly emergent influenza virus for detection of the newly emergent influenza virus without recompiling any of the independent learning algorithms. In embodiments, the emergent pathogen parameter definition file corresponds to a known target pathogen that has a genetic mutation; a newly discovered pathogen; a newly discovered pathogen strain; or a newly discovered pathogen subtype.

In some embodiments, for example, the provided method further comprises a step of independently verifying and validating the emergent pathogen parameter definition file and providing the independently verified and validated emergent pathogen parameter definition file to one or more of the sample analyzers. In embodiments, a plurality of emergent pathogen parameter definition files are continuously updated to provide characterization of newly emergent pathogens that otherwise are not characterized by the independent learning algorithm. In embodiments, the continuous update is by an automated update, a forced update, electronic email transmission to a user, a user download from a website or file transfer protocol, or through a cloud-based server or database. In an embodiment, the sample analyzer is configured to characterize a newly emergent pathogen without updating any of the independent learning algorithms.

In an embodiment, the method identifies an uncharacterized profile, further comprising the step of providing the uncharacterized profile to a third party for use in identifying a newly emergent pathogen and developing one or more emergent pathogen parameter definition files for the newly emergent pathogen. In embodiments, the third-party is an owner of the analyzer, a seller of the analyzer, a government organization, or a commercial pathogen characterization company.

In an embodiment, the independent learning algorithms are hard coded and cannot be edited by a user and the emergent pathogen parameter definitions files are read by the independent learning algorithms. In embodiments, the independent learning algorithms and the emergent pathogen parameter definitions files are integrated with the sample analyzer. In some embodiments, the independent learning algorithms and the emergent pathogen parameter definitions files are integrated in a separate component that receives the profile from the sample analyzer.

In an aspect, provided is a method for characterizing a target pathogen comprising: (i) providing a microarray having a plurality of capture sequences; (ii) contacting the microarray with a sample derived from a material potentially containing the target pathogens, wherein analytes from the target pathogen in the sample bind to at least a portion of the plurality of capture sequences; (iii) generating an intensity profile map corresponding to the microarray contacted with the sample and providing the intensity profile map to an analyzer; (iv) analyzing the intensity profile map using a plurality of independent learning algorithms and one or more emergent pathogen parameter definition files, wherein: each of the independent learning algorithms is taught and compiled using a profile corresponding to one or more known pathogens; at least a portion of the independent learning algorithms independently provide: (a) a known pathogen parameter output of the target pathogen based on the one or more known pathogen parameters; and (b) an emergent pathogen parameter output of the target pathogen based on the one or more emergent pathogen parameter definition files, wherein the emergent pathogen parameter output occurs without recompiling any of the learning algorithms; wherein the emergent pathogen definition files allow the independent learning algorithms to provide the emergent pathogen output without recompiling the learning algorithms or altering the known pathogen parameter outputs; and (v) combining the known pathogen parameter outputs and emergent pathogen parameter outputs for at least a portion of the independent learning algorithms to make a pathogen determination, thereby characterizing the one or more target pathogens.

In an aspect, provided is a pathogen characterization device comprising: (i) an imaging device for capturing an intensity profile map from a microarray that has been exposed to a material potentially containing a pathogen; an analyzer having a plurality of independent learning algorithms and emergent pathogen profile definition files, wherein (a) each of the independent learning algorithms is taught and compiled using a profile corresponding to one or more known pathogens; b) at least a portion of the independent learning algorithms independently provide: a known pathogen parameter output of the target pathogen based on the one or more known pathogen parameters; and an emergent pathogen parameter output of the target pathogen based on the one or more emergent pathogen parameter definition files, wherein the emergent pathogen parameter output occurs without recompiling any of the learning algorithms; and c) wherein the emergent pathogen definition files allow the independent learning algorithms to provide the emergent pathogen parameter output without recompiling the learning algorithms or altering the know pathogen parameter outputs; wherein the analyzer combines the known pathogen parameter outputs and emergent pathogen parameter outputs for at least a portion of the independent learning algorithms to make a determination, thereby characterizing the pathogen.

Provided below are exemplary claims:

1. A method for characterizing a target pathogen comprising: providing a sample derived from material potentially containing said target pathogen to a sample analyzer; generating a profile corresponding to said sample from said sample analyzer; analyzing said profile using a plurality of independent learning algorithms and one or more emergent pathogen parameter definition files, wherein: each of said independent learning algorithms is taught and compiled using a profile corresponding to one or more known pathogens; at least a portion of said independent learning algorithms independently provide: a known pathogen parameter output of said target pathogen based on said one or more known pathogen parameters; and an emergent pathogen parameter output of said target pathogen based on said one or more emergent pathogen parameter definition files, wherein said emergent pathogen parameter output occurs without recompiling any of said learning algorithms; wherein said emergent pathogen definition files allow said independent learning algorithms to provide said emergent pathogen parameter output without recompiling said learning algorithms or altering said known pathogen parameter outputs; and combining said known pathogen parameter outputs and emergent pathogen parameter outputs to make a pathogen determination, thereby characterizing said one or more target pathogens.

2. The method of claim 1, wherein said profile is an intensity profile map, a mass spectroscopy spectrum, an amino acid sequence or a nucleotide sequence.

3. The method of claim 2, wherein said profile is an intensity profile map obtained from a microarray having a plurality of capture sequences, each capture sequence configured to bind to a target sequence of interest.

4. The method of any of claims 1-3, wherein said characterization is an identification of the presence or absence of said target pathogen.

5. The method of any of claims 1-4, wherein said characterization is an identification of one or more pathogen parameters of said target pathogen indicative of an emergent pathogen, corresponding to an unknown or null value of one or more of said known pathogen parameters.

6. The method of any of claims 1-5, wherein said pathogen parameters are selected from the group consisting of: type, subtype, genotype, absence of pathogen, strain, lineage, seasonality, mutation presence or absence, marker presence or absence, virulence, novelty, species of origin and any combination thereof.

7. The method of any of claims 1-6, wherein each of said independent learning algorithms is independently trained to evaluate a single pathogen parameter of a target pathogen.

8. The method of any of claims 1-7, wherein at least a portion of said independent learning algorithms are independent artificial neural network (ANN) algorithms.

9. The method of any of claims 1-8, wherein said independent learning algorithms are supervised learning algorithms.

10. The method of any of claim 9, wherein at least a portion of said supervised learning algorithms are selected from the group consisting of: a support vector machine; a decision tree; a clustering algorithm, a Bayesian network, a random forest, a logistic regression algorithm, a K-nearest neighbor algorithm, and any combination thereof.

11. The method of any of claims 1-10, wherein said target pathogen is one or more influenza viruses.

12. The method of any of claims 1-11, wherein said known pathogen parameters correspond to one or more of influenza A, influenza B, influenza A seasonal H1N1 viral strains, influenza A seasonal H3N2 viral strains, or influenza A non-seasonal viral strains.

13. The method of any of claims 1-12, wherein said known pathogen parameters correspond to one or more of: H1, H2, H3, H5, H7 and H9 hemagglutinin subtypes and N1, N2, N7, N8 and N9 neuraminidase subtypes.

14. The method of claim 13, wherein said target pathogen is influenza A and at least one of said plurality of independent learning algorithms provides outputs corresponding to HA subtype and at least one of said plurality of independent learning algorithms provides outputs corresponding to NA subtype.

15. The method of any of claims 1-14, wherein said sample is a material potentially containing said pathogen is a biological material from a human or a non-human animal, an isolate or a culture.

16. The method of any of claims 1-15, wherein said sample potentially contains one or more influenza viruses.

17. The method of any of claims 1-16, wherein said sample analyzer is a microrarray, a genetic sequencer, a protein sequencer or a mass spectrometer.

18. The method of claim 17, wherein said sample analyzer is a microarray.

19. The method of any of claims 1-18, wherein said emergent pathogen parameter definition file is periodically provided to said sample analyzer.

20. The method of any of claims 1-19, wherein said emergent pathogen parameter definition file corresponds to a newly emergent influenza virus for detection of said newly emergent influenza virus without recompiling any of said independent learning algorithms.

21. The method of any of claims 1-20, wherein said emergent pathogen parameter definition file corresponds to a known target pathogen that has a genetic mutation; a newly discovered pathogen; a newly discovered pathogen strain; or a newly discovered pathogen subtype.

22. The method of any of claims 1-21 further comprising a step of independently verifying and validating said emergent pathogen parameter definition file and providing said independently verified and validated emergent pathogen parameter definition file to one or more of said sample analyzers.

23. The method of any of claims 1-22, wherein a plurality of emergent pathogen parameter definition files are continuously updated to provide characterization of newly emergent pathogens that otherwise are not characterized by said independent learning algorithm.

24. The method of claim 23, wherein said continuous update is by an automated update, a forced update, electronic email transmission to a user, a user download from a website or file transfer protocol, or through a cloud-based server or database.

25. The method of any of claims 1-24, wherein said sample analyzer is configured to characterize a newly emergent pathogen without updating any of said independent learning algorithms.

26. The method of any of claims 1-25, that identifies an uncharacterized profile, further comprising the step of providing said uncharacterized profile to a third party for use in identifying a newly emergent pathogen and developing one or more emergent pathogen parameter definition files for said newly emergent pathogen.

27. The method of claim 26, wherein said third-party is an owner of the analyzer, a seller of the analyzer, a government organization, or a commercial pathogen characterization company.

28. The method of any of claims 1-27, wherein said independent learning algorithms are hard coded and cannot be edited by a user and the emergent pathogen parameter definitions files are read by said independent learning algorithms.

29. The method of any of claims 1-28, wherein said independent learning algorithms and said emergent pathogen parameter definitions files are integrated with the sample analyzer.

30. The method of any of claims 1-28, wherein said independent learning algorithms and said emergent pathogen parameter definitions files are integrated in a separate component that receives said profile from said sample analyzer.

31. A method for characterizing a target pathogen comprising: providing a microarray having a plurality of capture sequences; contacting said microarray with a sample derived from a material potentially containing said target pathogens, wherein analytes from said target pathogen in said sample bind to at least a portion of said plurality of capture sequences; generating an intensity profile map corresponding to said microarray contacted with said sample and providing said intensity profile map to an analyzer; analyzing said intensity profile map using a plurality of independent learning algorithms and one or more emergent pathogen parameter definition files, wherein: each of said independent learning algorithms is taught and compiled using a profile corresponding to one or more known pathogens; at least a portion of said independent learning algorithms independently provide: a known pathogen parameter output of said target pathogen based on said one or more known pathogen parameters; and an emergent pathogen parameter output of said target pathogen based on said one or more emergent pathogen parameter definition files, wherein said emergent pathogen parameter output occurs without recompiling any of said learning algorithms; wherein said emergent pathogen definition files allow said independent learning algorithms to provide said emergent pathogen output without recompiling said learning algorithms or altering said known pathogen parameter outputs; and combining said known pathogen parameter outputs and emergent pathogen parameter outputs for at least a portion of said independent learning algorithms to make a pathogen determination, thereby characterizing said one or more target pathogens.

32. The method of claim 31, wherein said characterization is an identification of the presence or absence of said target pathogen.

33. The method of any of claim 31 or 32, wherein said characterization is an identification of one or more pathogen parameters of said target pathogen indicative of an emergent pathogen, corresponding to an unknown or null value of one or more known pathogen parameters.

34. The method of any of claims 31-33, wherein said pathogen parameters are selected from the group consisting of: type, subtype, genotype, absence of pathogen, strain, lineage, seasonality, mutation presence or absence, marker presence or absence, virulence, novelty, species of origin and any combination thereof.

35. The method of any of claims 31-34, wherein each of said independent learning algorithms is independently trained to evaluate a single pathogen parameter of a target pathogen.

36. The method of any of claims 31-35, wherein at least a portion of said independent learning algorithms are independent artificial neural network (ANN) algorithms.

37. The method of any of claims 31-36, wherein said independent learning algorithms are supervised learning algorithms.

38. The method of claim 37, wherein at least a portion of said independent supervised learning algorithms are selected from the group consisting of: a support vector machine; a decision tree; a clustering algorithm, a Bayesian network, a random forest, a logistic regression algorithm, a K-nearest neighbor algorithm, and any combination thereof.

39. The method of any of claims 31-38, wherein said target pathogen is one or more influenza viruses.

40. The method of any of claims 31-39, wherein said known pathogen parameters correspond to one or more of influenza A, influenza B, influenza A seasonal H1N1 viral strains, influenza A seasonal H3N2 viral strains, or influenza A non-seasonal viral strains.

41. The method of any of claims 31-40, wherein said known pathogen parameters correspond to one or more of: H1, H2, H3, H5, H7 and H9 hemagglutinin subtypes and N1, N2, N7, N8 and N9 neuraminidase subtypes.

42. The method of claim 41, wherein said target pathogen is influenza A and at least one of said plurality of independent learning algorithms provides outputs corresponding to HA subtype and at least one of said plurality of independent learning algorithms provides outputs corresponding to NA subtype.

43. The method of any of claims 31-42, wherein said emergent pathogen parameter definition file is periodically provided to said analyzer.

44. The method of any of claims 31-43, wherein said emergent pathogen parameter definition file corresponds to a newly emergent influenza virus for detection of said newly emergent influenza virus without recompiling any of said independent learning algorithms.

45. The method of any of claims 31-44, wherein said emergent pathogen parameter definition file corresponds to a known target pathogen that has a genetic mutation; a newly discovered pathogen; a newly discovered pathogen strain; or a newly discovered pathogen subtype.

46. The method of any of claims 31-45 further comprising the step of independently verifying and validating said emergent pathogen parameter definition file and providing said independently verified and validated emergent pathogen parameter definition file to one or more of said analyzers.

47. A pathogen characterization device comprising: an imaging device for capturing an intensity profile map from a microarray that has been exposed to a material potentially containing a pathogen; an analyzer having a plurality of independent learning algorithms and emergent pathogen profile definition files, wherein a) each of said independent learning algorithms is taught and compiled using a profile corresponding to one or more known pathogens; b) at least a portion of said independent learning algorithms independently provide: a known pathogen parameter output of said target pathogen based on said one or more known pathogen parameters; and an emergent pathogen parameter output of said target pathogen based on said one or more emergent pathogen parameter definition files, wherein said emergent pathogen parameter output occurs without recompiling any of said learning algorithms; and c) wherein said emergent pathogen definition files allow said independent learning algorithms to provide said emergent pathogen parameter output without recompiling said learning algorithms or altering said know pathogen parameter outputs; wherein said analyzer combines said known pathogen parameter outputs and emergent pathogen parameter outputs for at least a portion of said independent learning algorithms to make a determination, thereby characterizing said pathogen.

### BRIEF DESCRIPTION OF THE DRAWINGS

FIG. 1 provides a flow diagram of a method for characterizing a pathogen using both a diagnostic mode (left) and an open mode (right).

FIG. 2 provides a flow diagram illustrating the multiple sets of definition files as well as outputs.

FIG. 3 shows the interaction between learning algorithms (in this embodiment, artificial neural networks (ANNs)) and the definition files.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Pathogen” refers to an infectious agent such as a virus or bacterium. Target pathogen refers to a pathogen in a sample under analysis, for example, having specific characteristics, such as type, subtype, genotype, absence of pathogen, strain, lineage, or seasonality. The present methods and systems are useful for determining the presence, absence and/or characteristics of target pathogens in a sample.

“Supervised learning” is a subset of machine learning algorithms, within the field of pattern recognition. “Supervised learning algorithm” is an algorithm that utilizes supervised learning for the purpose of identifying and/or characterizing features in an input, such as in microarray data. In some embodiments, supervised learning algorithms of the invention identify and/or characterize features in microarray data corresponding to a target pathogen such as a pathogen parameter, for example, a comparison of output from an unknown sample to an expected output for known and well-characterized pathogens. “Independent supervised learning algorithms” refers to a plurality of supervised learning algorithms that operate independently to receive and analyze microarray data, for example, so as to provide outputs corresponding to pathogen parameters. Independent supervised learning algorithms may operate in parallel or in sequence. A plurality of independent supervised learning algorithms that are trained using microarray data for known samples may be used. Accordingly, there is a combined output from the plurality of independent supervised learning algorithms to provide a determination, such as indicating the presence or absence of a target pathogen, characterizing features of a target pathogen, or otherwise providing diagnostically relevant information.

“Unsupervised learning” (or “Unstructured learning”) is also a subset of machine learning algorithms, within the field of pattern recognition. Unsupervised learning algorithm is an algorithm that utilizes unsupervised learning for the purpose of identifying and/or characterizing new or previously unrecognized features in a dataset, such as in microarray data. In some embodiments, unsupervised learning algorithms of the invention identify and/or characterize features in microarray data corresponding to a new or emerging target pathogen (such as a pathogen parameter) for which prior identified patterns are not available. In some embodiments, unsupervised learning in the form of cluster analysis is performed to identify a group of samples that correspond to an emergent pattern. Supervised learning can then be used to develop new algorithms to identify the emergent pattern in subsequent data.

“Pathogen parameter” refers to a characteristic or feature of a pathogen, such as a target pathogen. Pathogen parameters may include the presence or absence of a target pathogen. Pathogen parameters may include type, subtype, genotype, absence of pathogen, strain, lineage, seasonality, host species adaptation, presence or absence of a mutation, or presence or absence of a marker. Pathogen parameter also may refer to whether a pathogen is known (i.e. previously classified) or emergent. In the context of influenza target pathogens, for example, pathogen parameters include identification or classification of influenza A, influenza B, influenza A seasonal H1N1 subtype, influenza A seasonal H3N2 subtype, influenza A non-seasonal subtype, H5N1 subtype, H5N2 subtype, H7N9 subtype, H9N2 subtype, H3N8 subtype, individual HA subtypes (including, for example, H1, H3, H5, H7 & H9), individual NA subtypes (including, for example, N1, N2, N7, N8 and N9), pathogenicity marker, and putative antiviral resistance markers such as 275Y NA mutation, 119V NA mutation, 292K NA mutation or 155H NA mutation.

“Known pathogen” refers to a pathogen strain that has been previously identified and classified. In some embodiments, known pathogens have an established profile, for example a microarray intensity profile, which have been previously introduced into the analysis system, thereby allowing for accurate recognition. For example, known pathogens may be pathogens that had established profiles at the time the analysis software was compiled. In this manner, the known pathogen may be considered well-characterized in terms of an expected output, or microarray intensity profile, for the microarray. For test samples having similar outputs, the supervised learning algorithms and/or compiled algorithms may then infer pathogen type by characterizing the output as related to a known output. Substantial deviations from any known output may reflect a test sample containing an emergent pathogen.

“Emergent pathogen” refers to a pathogen strain or mutation that has been newly discovered or has recently mutated from a known pathogen. In embodiments, for example, emergent pathogens do not have established profiles, for example a microarray intensity profile, and cannot be accurately characterized by an un-updated analyzer. In some embodiments, emergent pathogens are those pathogens which have been discovered after the analysis software has been compiled.

“Pathogen Parameter Definition File” refers to data corresponding to a pathogen parameter used by the learning algorithm to make future characterizations of unknown pathogens. In embodiments, for example, the pathogen parameter definition file may refer to a set of intensity profiles, weighting factors, algorithm configuration parameters, or other mathematical operations corresponding to microarrays corresponding to pathogens having known or emergent strains or pathogen parameters.

“Emergent Pathogen Parameter Definition File” refers to a pathogen parameter definition file corresponding to a new or unknown emergent pathogen. In some embodiments, emergent pathogen parameter definition files are used by learning algorithms to determine if an unknown pathogen has been previously characterized or analyzed.

“Sample” refers to a composition derived from a material, such as a material potentially containing target pathogens. Embodiments of the present methods are useful for analyzing samples derived from a wide range of materials including clinical samples, biological material from a human or a non-human animal, an environmental material that is suspected of containing influenza, a material grown in cell culture or an egg culture or grown by other methods. In some embodiments, a sample is derived by processing a material potentially containing target pathogens, such as processing involving extraction, amplification, fragmentation and/or purification of biological materials such as oligonucleotides and nucleic acids.

“Unknown value” or “null value” are used interchangeably and refer to a condition in which the algorithm cannot accurately determine the desired pathogen parameter. An unknown or null value may indicate that the target pathogen cannot be accurately characterized or compared to a known pathogen, and thus, may reflect that the target pathogen is an emergent pathogen. For example, when characterizing a viral strain of a target pathogen, an unknown value may indicate that the target pathogen does not correspond to a known strain or mutated strain.

Aspects of the invention provide methods for processing and/or analyzing microarray data. The method is useful for rapidly identifying specific types, subtypes and/or strains of pathogenic infections present in clinical samples, isolates, or other samples suspected of containing pathogens. Methods provided herein may use the intensities of various oligonucleotide capture sequences on a microarray as inputs to predict which type or subtype of pathogen is present using a mathematical model that utilizes supervised learning.

Supervised learning algorithms may employ machine learning to learn from and make predictions based on complex data. More specifically these types of algorithms operate by constructing a mathematical model from example data that can be used to make predictions or decisions based on novel data. Supervised learning algorithms, which are employed in the invention, for example, may infer a predictive model from a “training” data set that consists of example input values paired with expected output values. Input values may consist of any pre-defined set of quantifiable features that can be extracted from each object presented to the algorithm. Output values can be associated with labeled categories, scores or other known characteristics of each object. The goal of the training phase to is generalize a function, or set of functions, that can then be used to recognize unseen and unique feature sets and determine their similarity to the objects presented during training. Output values correspond to the labels or classifications attributed to those known objects. In this manner, algorithms may be constructed to make broad or very specific classifications or decisions depending on the composition of the representative training set, number of outputs and the degree of function generalization.

Well-characterized samples that represent each different “category” or “class” of the pathogen to be identified (e.g., types, subtypes, serotypes, strains, etc.) are subjected to nucleic acid extraction, the nucleic acid amplified, hybridized to a microarray, and imaged to generate an array of fluorescence intensities (for each capture sequence) utilized for training. In embodiments, samples containing other pathogens and samples containing no pathogens but containing human genetic material are also processed to generate microarray patterns for training as clinical negatives. Microarray data from these well-characterized samples form a dataset that is used to train a set of pattern recognition algorithms to recognize the features of the various categories/classes, and those of clinical negatives.

Numerous “building block” algorithms may be individually trained to identify different classes or categories of the pathogen. Examples include a block to identify pathogen type (e.g., that may represent multiple subtypes that are all categorized as the same type), a specific pathogen subtype, or patterns wherein the target pathogen is not present (although other potentially interfering pathogens may be). The features used as inputs to the algorithms are typically some statistical measure of spot intensities (such as the mean or median spot intensities) collected for each capture sequence. Each building block may output a value between 0 and 1, where a value closer to 1 indicates that the pattern of intensities for the unknown sample in question matches closely the pattern for the training set, and a value closer to 0 indicates the unknown sample in question does not match the pattern for the training set. The various building blocks are then linked together logically in order to make a final determination of the pathogen detection, for example, via a logical cascade architecture relating to the categories and subcategories of pathogen parameters. In embodiments, thresholds, for example as defined as the value between 0 and 1 that is used to distinguish between a “positive” and “negative” call, are chosen for each of the blocks in order to optimize the performance of the system as a whole.

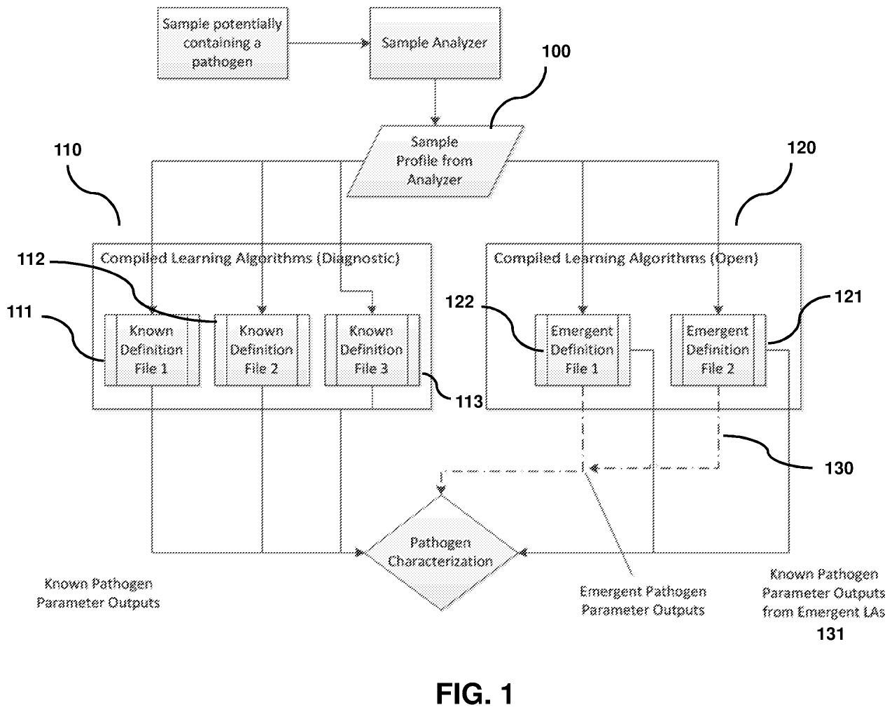

FIG. 1 is a flow chart summary of one method for characterizing a pathogen with two separate modes: a diagnostic mode in which all definition files are derived from known, well-characterized pathogens and an open mode in which emergent pathogen definition files may also be accessed by the learning algorithms. First, a sample potentially containing a pathogen is introduced into an analyzer in order to generate a pathogen profile 100. The profile is then analyzed by the compiled learning algorithms. In the case of diagnostic mode 110 (illustrated on the left), the complied algorithms only have access to known and well-characterized definition files 111, 112, 113. Thus, all pathogen parameter outputs from the sample are compared against known output and the pathogen characterization only makes characterizations according to well-characterized pathogen profiles.

In contrast, in open mode 120, the learning algorithms may also access and utilize emergent pathogen definition files 121, 122. These files include pathogens that have been recently discovered but not yet fully studied and definition files that allow one or more the algorithms to make an “unknown or null” determination 130 (reflected by the dashed line), in which the sample pathogen is characterized as emergent and flagged for further study. The emergent definition files 121, 122 may also make characterizations that the sample pathogen is similar to a recently discovered emergent pathogen 131. While this characterization may not be as well-established as a determination from diagnostic mode 110, it may still be helpful in providing researchers with data regarding a potential new viral strain or mutation, as well as identifying cases of potential viral outbreaks.

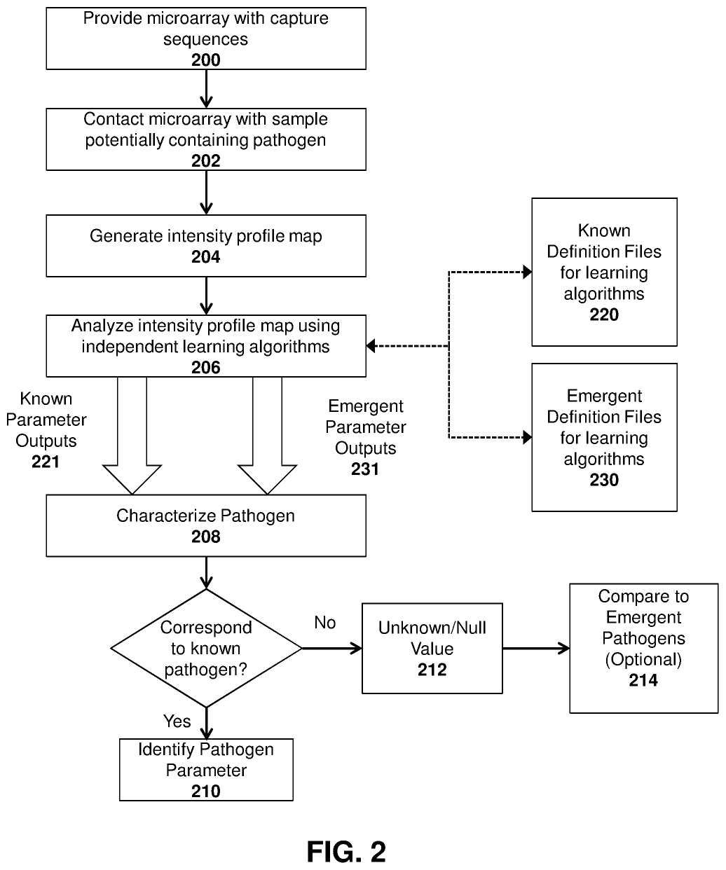

FIG. 2 provides a flow diagram of a method for pathogen detection utilizing microarrays and analyzing with both known and emergent pathogen definition files. First, a microarray with a plurality of capture sequences 200 is contacted with a sample potentially containing a pathogen, including a sample that has been processed to provide accessible targets from a pathogen 202. The process may be, for example, polymerase chain reaction (PCR) using a variety of primers selected to target genetic regions of interest for a range of pathogens, including influenza. Next a microarray intensity profile is generated corresponding to the sample 204. Then the sample intensity profile is analyzed by a plurality of learning algorithms which use definition files to make characterization of the sample intensity profile 208. In this case, the algorithms have access to both known 220 and emergent 230 pathogen parameter definition files. The algorithms then provide known parameter outputs and some may also provide emergent parameter outputs which suggest that the sample is either unknown, emergent, or corresponds more closely to an emergent pathogen previously identified and placed or updated in the emergent definition files. The step of identification 210 then can include: (1) identifying the pathogen as a known, characterized strain or trait, (2) identifying the pathogen or pathogen trait as unknown or emergent and/or (3) recognizing the pathogen or pathogen trait as being related or similar to another recently analyzed unknown or emergent strain. In the case of an unknown or emergent strain, the emergent parameter outputs 231 may be optionally analyzed to compare to other pathogens which have been characterized as emergent 214.

### Example 1—Plug-in Expertise for Pathogen Identification Using Modular Neural Network Architecture

Methods provided herein facilitate dynamic runtime use and configuration of one or many algorithms for sensor data processing and pathogen identification without requiring a modification of underlying software source code, including software compilation. In an embodiment, system software retrieves configuration files from a location in local storage or from a network and uses those files to determine relevant input and output parameters for machine learning algorithms to process incoming data sets.

A traditional compile-time defined algorithm is created using source code that is compiled into the “final” executable software package that will be distributed to end users. It can be said that this approach is “hard-coded”, meaning that the structure and operation of the algorithm are defined up-front and immutable as long as the software package is unchanged. In contrast, a runtime defined algorithm is newly created (“interpreted” rather than compiled), each time the software package is launched. Parameters that control the creation of the algorithm, and determine its structure and purpose, reside external to the software package and are accessed by the software while it is running.

The concept of runtime defined algorithms or process flows is known in the field of computer science. However, employing these methods in a pathogen detection system confers numerous benefits. Hard-coded algorithms or parameters limit the use of a system to purposes and techniques that can be identified during the initial development of the software. In the real-world pathogens evolve, new ones emerge and techniques are refined. The modular approach provided herein allows the system to adapt to these changing conditions in order to improve accuracy and detect new pathogens or pathogen mutations. This provides numerous benefits including 1) identification of previously unknown patterns, such as genetic information of emergent viruses, 2) the testing of new parameter sets for their ability to identify existing or emergent pathogens or patterns without modifying the software source code or existing parameter sets, 3) the ability to test updated parameter sets for increased accuracy of identification without modifying the source code or existing parameter sets, and 4) the ability to leave the diagnostic mode that requires regulatory clearance undisturbed while testing new or updated parameter sets.

Hard-coded algorithms are inherently tied to the software release version. In order to update them, new software must be developed, tested, and distributed to end users for installation. In contrast, runtime defined algorithm files provided herein can be updated and used immediately by the software system without requiring risky or error-prone software updates to be performed by the end user.

The provided methods allow for rapid system adaptation to changing pathogen profiles and potential pandemic outbreaks. Target pathogens and their biological features can change and evolve over time. Additionally, entirely new pathogens or pathogen strains/subtypes are periodically discovered or rapidly emerge with attendant pandemic outbreak concerns. Machine learning algorithms, one family of algorithms used in an embodiment of the method, may improve in accuracy over time as more information is collected about target pathogens. The ability to rapidly deploy new and/or updated algorithms greatly enhances the robustness and utility of a software system that is designed to detect pathogens.

Because software systems are inherently complex, new versions of software require rigorous testing at the unit and system level. Limiting the scope of the changes to files and methods external to the main system software reduces the risk of bugs or unintended side-effects that are common when updating software source code. Runtime defined algorithms can be separately verified and validated outside of the hard-coded software system increasing result confidence and offering more robust test methods and protocols.

Regulatory Oversight: Due to the risk inherent in software updates, regulatory bodies may require new or updated submissions and regulatory approval in order to release new or updated versions of software. Submitting updates for approval is not only costly but time consuming, thereby slowing down the release of potentially relevant data corresponding to new strains or mutations. In one embodiment, algorithms approved for diagnostic use are compiled into the software. The same software can be used in “Open” mode to obtain additional non-diagnostic information for research purposes provided by the runtime defined algorithms. Because of the inherent separation provided by runtime defined algorithms, the software can take advantage of continuous updates to those algorithms while maintaining a static codebase maintained under strict regulatory controls.

Therefore, by allowing both compiled and run-time modes of use and continual improvement in analysis capabilities from a single software package, detection capabilities, pandemic recognition, development time, support efforts, and production/distribution activities may be significantly reduced compared to similar systems that require distinct software versions and updates.

### Example 2—Artificial Neural Networks for Identifying and Characterizing Influenza

In one embodiment, a software application is designed to provide influenza diagnostic and subtyping capabilities by analyzing data from a patient sample. The application may run in one of two modes: (1) The Clinical Mode which provides clinically relevant and FDA-approved diagnostic results and (2) The Open mode augments the information provided in the clinical mode with extra content regarding the patient sample that is not approved or intended for use in patient diagnosis. This extra information is useful in public-health and research settings and has the potential to realize valuable contributions to the understanding of influenza and its epidemiology.

The software application may be written in C #, and use DNA microarray data collected from a florescence imager to interpret intensity values from a series of target oligonucleotides. These intensity values provide a unique “fingerprint” for each sample. While each of these fingerprints is slightly different and unique to each patient sample and potential influenza strain, they all share similar features. These similarities are used by trained machine learning algorithms to classify the data into multiple categories. Artificial Neural Networks (ANNs) may be the primary algorithm employed to make these classifications. The application may employ the open-source FANN Library (http://leenissen.dk/) to implement each of the classifiers. However, there are many families and types of machine learning algorithms that may be used in place of ANNs or to augment them. For example, Support Vector Machines (SVMs), Decision Trees, Boosting or Regression, among many others.

The application may use configuration files or definition files to setup, configure, or define each of the ANNs. For the clinical classifications, these files may be compiled into the software, essentially hard-coding them into that software release. For the open mode classifications, the software scans a location on the local hard drive when it is launched. For each of the valid definition files found, the software creates a neural network using those parameters. Thus, when a sample is analyzed in open mode, each of those ANNs may be used to provide additional classifications for that sample. The result, name of the network and checksum for the definition file is stored to provide traceability back to the file that was used to perform the analysis.

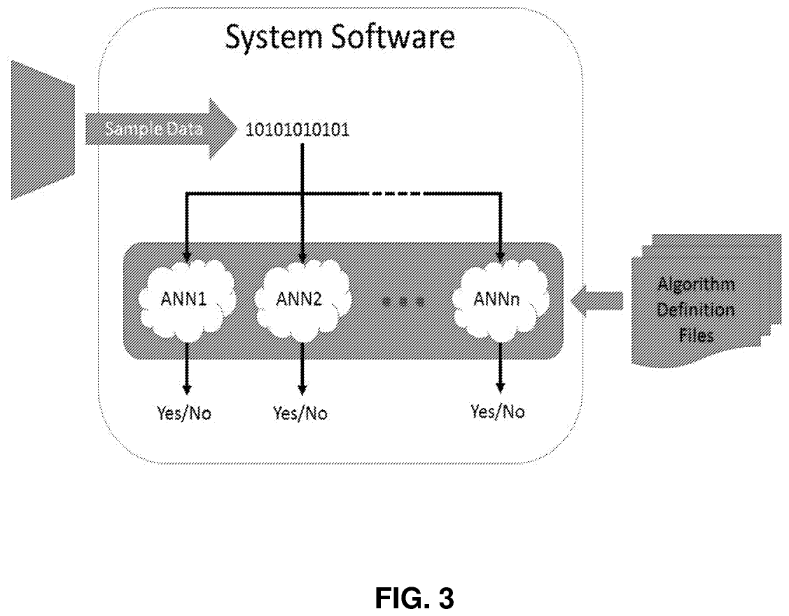

FIG. 3 illustrates how algorithm definition files may be read into the system software and used for analysis. The definition files are independent of the software system and can be stored and updated without impacting the software in any way, including locally or externally, such as automatic update from a web-based server.

This unique modular approach enables rapid “plug-in” expertise for identification/classification of new pathogens. They system provides substantial commercial benefits to both the system provider and laboratory operators. For example, when a new pathogen, such as influenza virus, emerges a new definition file maybe independently developed by the manufacturer and electronically distributed to operators. Once the operator accesses the new definition file, the system can identify that new pathogen without any impact on the overall system or software. This modularity provides a more efficient and rapid means for identifying emergent pathogens or pathogen mutations than any other diagnostic systems, avoiding the need for timely software overhauls or potential regulatory review.

Relevant Influenza Virus Background: The methods and systems may be used to identify types and subtypes of influenza virus. Influenza virus belongs to the virus family Orthomyxoviridae and consists of an 8-piece segmented RNA genome that codes for 11 proteins. The segmented RNA genome makes the influenza virus prone to mutations, both due to errors in RNA replication (antigenic drift, which gives rise to seasonal epidemics) and drastic changes in the viral genome due to reassortment of genetic segments from different parent viruses (antigenic shift, which gives rise to pandemics). Influenza A viruses historically give rise to both epidemics and pandemics, whereas influenza B viruses give rise to only seasonal epidemics.

The types of influenza virus known to cause regular infections in humans and animals are referred to as A and B. Influenza type B is not as genetically diverse as influenza A, and is characterized by two different lineages (the Yamagata lineage and the Victoria lineage) based on phylogeny. In addition, influenza B mainly infects humans.

Influenza type A consists of a variety of subtypes, based on the makeup of the two surface proteins, hemagglutinin (HA) and neuraminidase (NA). There are currently 16 known HA subtypes and 9 known NA subtypes that combine in a variety of ways, giving rise to the standard HXNY nomenclature (ex: H3N2, H5N1). All influenza A viral subtypes have been isolated from wild aquatic birds (the natural reservoir of influenza virus), but infections occur in other animal species including humans. The most common influenza A subtypes infecting humans are H1, H2, H3, N1, and N2.

The currently circulating seasonal subtypes of influenza A are H1N1 and H3N2. “Non-seasonal” subtypes of influenza A (defined as those subtypes that are not seasonal H1N1 or seasonal H3N2) are numerous, and include but are not limited to many subtypes of higher prevalence in animals and/or potentially pandemic importance such as H5N1, H5N2, H7N9, H7N2, H7N3, H9N2, H7N7, H3N8, and H1N1 of swine and avian origin.

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Specific names of compounds or components are intended to be exemplary, as it is known that one of ordinary skill in the art can name the same compounds or components differently.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

US Patent Publication no. 2009/0124512
US Patent Publication no. 2010/0130378
US Patent Publication no. 2010/0273670
US Patent Publication no. 2014/0221234
Heil, G L, McCarthy, T, Yoon, K-J, Darwish, M, Smith, C B, Houck, J A, Dawson, E D, Rowlen, K L, Gray, G C “MChip, a low density microarray, differentiates among seasonal human H1N1, classical swine H1N1, and the 2009 pandemic H1N1”, Influenza Other Respir Viruses 2010, 4(6), 411-416.
Townsend, M B, Smagala, J A, Dawson, E D, Deyde, V, Gubareva, L, Klimov, A I, Kuchta, R D, Rowlen, K L, “Detection of Adamantane-Resistant Influenza on a Microarray”, J Clin Viro/2008, 42(2), 117-123.
Moore, C L, Smagala, J A, Smith, C B, Dawson, E D, Cox, N J, Kuchta, R D, Rowlen, K L “Evaluation of MChip with Historic A/H1N1 Influenza Viruses Including the 1918 “Spanish Flu'”J Clin Microbiol 2007, 45(11), 3807-3810.
Mehlmann, M, Bonner, A B, Williams, J V, Dankbar, D M, Moore, C L, Kuchta R D, Podsiad, A B, Tamerius, J D, Dawson, E D, Rowlen, K L “Comparison of the MChip to Viral Culture, Reverse Transcription-PCR, and the QuickVue Influenza A+B Test for Rapid Diagnosis of Influenza” J Clin Microbiol 2007, 45: 1234-1237.
Dankbar, D M, Dawson, E D, Mehlmann, M, Moore, C L, Smagala, J A, Shaw, M W, Cox, N J, Kuchta, R D, Rowlen, K L. “Diagnostic microarray for influenza B viruses” Anal Chem 2007, 79(5), 2084-2090.
Dawson, E D, Moore, C L, Dankbar, D M, Mehlmann, M Townsend, M B, Smagala, J A, Smith, C B, Cox, N J, Kuchta, R D, Rowlen, K L “Identification of A/H5N1 influenza viruses using a single gene diagnostic microarray” Anal Chem 2007, 79(1), 378-384.
Dawson, E D, Moore, C L, Smagala, J A, Dankbar, D M, Mehlmann, M Townsend, M B, Smith, C B, Cox, N J, Kuchta, R D, Rowlen, K L “MChip: A tool for influenza surveillance” Anal Chem 2006, 78(22), 7610-7615.
Dawson, E D, Rowlen, K L “MChip: A Single Gene Diagnostic for Influenza A”, in Influenza: Molecular Virology, Wang, Q. and Tao, Y. J., eds. (Norfolk, U K, Caister Academic Press), February 2010, book chapter.

1. A method for characterizing a target pathogen comprising:
providing a sample derived from material potentially containing said target pathogen to a sample analyzer;
generating a profile corresponding to said sample from said sample analyzer;
analyzing said profile using a plurality of independent learning algorithms and one or more emergent pathogen parameter definition files, wherein:
each of said independent learning algorithms is taught and compiled using a profile corresponding to one or more known pathogens;
at least a portion of said independent learning algorithms independently provide:
a known pathogen parameter output of said target pathogen based on said one or more known pathogen parameters; and
an emergent pathogen parameter output of said target pathogen based on said one or more emergent pathogen parameter definition files, wherein said emergent pathogen parameter output occurs without recompiling any of said learning algorithms;
wherein said emergent pathogen definition files allow said independent learning algorithms to provide said emergent pathogen parameter output without recompiling said learning algorithms or altering said known pathogen parameter outputs; and
combining said known pathogen parameter outputs and emergent pathogen parameter outputs to make a pathogen determination, thereby characterizing said one or more target pathogens.

2. The method of claim 1, wherein said profile is an intensity profile map, a mass spectroscopy spectrum, an amino acid sequence or a nucleotide sequence.

3. The method of claim 2, wherein said profile is an intensity profile map obtained from a microarray having a plurality of capture sequences, each capture sequence configured to bind to a target sequence of interest.

4. The method of claim 1, wherein said characterization is an identification of the presence or absence of said target pathogen.

5. The method of claim 1, wherein said characterization is an identification of one or more pathogen parameters of said target pathogen indicative of an emergent pathogen, corresponding to an unknown or null value of one or more of said known pathogen parameters.

6. The method of claim 1, wherein said pathogen parameters are selected from the group consisting of: type, subtype, genotype, absence of pathogen, strain, lineage, seasonality, mutation presence or absence, marker presence or absence, virulence, novelty, species of origin and any combination thereof.

7. The method of claim 1, wherein each of said independent learning algorithms is independently trained to evaluate a single pathogen parameter of a target pathogen, wherein at least a portion of said independent learning algorithms are independent artificial neural network (ANN) algorithms.

8. (canceled)

9. The method of claim 1, wherein said independent learning algorithms are supervised learning algorithms, wherein at least a portion of said supervised learning algorithms are selected from the group consisting of: a support vector machine; a decision tree; a clustering algorithm, a Bayesian network, a random forest, a logistic regression algorithm, a K-nearest neighbor algorithm, and any combination thereof.

10. (canceled)

11. The method of claim 1, wherein said target pathogen is one or more influenza viruses.

12. The method of claim 1, wherein said known pathogen parameters correspond to one or more of influenza A, influenza B, influenza A seasonal H1N1 viral strains, influenza A seasonal H3N2 viral strains, or influenza A non-seasonal viral strains.

13. The method of claim 1, wherein said known pathogen parameters correspond to one or more of: H1, H2, H3, H5, H7 and H9 hemagglutinin subtypes and N1, N2, N7, N8 and N9 neuraminidase subtypes.

14. The method of claim 13, wherein said target pathogen is influenza A and at least one of said plurality of independent learning algorithms provides outputs corresponding to HA subtype and at least one of said plurality of independent learning algorithms provides outputs corresponding to NA subtype.

15. The method of claim 1, wherein said sample is a material potentially containing said pathogen is a biological material from a human or a non-human animal, an isolate or a culture.

16. (canceled)

17. The method of claim 1, wherein said sample analyzer is a microrarray, a genetic sequencer, a protein sequencer or a mass spectrometer.

18. (canceled)

19. The method of claim 1, wherein said emergent pathogen parameter definition file is periodically provided to said sample analyzer.

20. The method of claim 1, wherein said emergent pathogen parameter definition file corresponds to a newly emergent influenza virus for detection of said newly emergent influenza virus without recompiling any of said independent learning algorithms.

21. The method of claim 1, wherein said emergent pathogen parameter definition file corresponds to a known target pathogen that has a genetic mutation; a newly discovered pathogen; a newly discovered pathogen strain; or a newly discovered pathogen subtype.

22. The method of claim 1 further comprising a step of independently verifying and validating said emergent pathogen parameter definition file and providing said independently verified and validated emergent pathogen parameter definition file to one or more of said sample analyzers.

23. The method of claim 1, wherein a plurality of emergent pathogen parameter definition files are continuously updated to provide characterization of newly emergent pathogens that otherwise are not characterized by said independent learning algorithm, wherein said continuous update is by an automated update, a forced update, electronic email transmission to a user, a user download from a website or file transfer protocol, or through a cloud-based server or database.

24. (canceled)

25. The method of claim 1, wherein said sample analyzer is configured to characterize a newly emergent pathogen without updating any of said independent learning algorithms.

26. The method of claim 1, that identifies an uncharacterized profile, further comprising the step of providing said uncharacterized profile to a third party for use in identifying a newly emergent pathogen and developing one or more emergent pathogen parameter definition files for said newly emergent pathogen.

27. (canceled)

28. The method of claim 1, wherein said independent learning algorithms are hard coded and cannot be edited by a user and the emergent pathogen parameter definitions files are read by said independent learning algorithms.

29. The method of claim 1, wherein said independent learning algorithms and said emergent pathogen parameter definitions files are integrated with the sample analyzer.

30. The method of claim 1, wherein said independent learning algorithms and said emergent pathogen parameter definitions files are integrated in a separate component that receives said profile from said sample analyzer.

31. A method for characterizing a target pathogen comprising:
providing a microarray having a plurality of capture sequences;
contacting said microarray with a sample derived from a material potentially containing said target pathogens, wherein analytes from said target pathogen in said sample bind to at least a portion of said plurality of capture sequences;
generating an intensity profile map corresponding to said microarray contacted with said sample and providing said intensity profile map to an analyzer;
analyzing said intensity profile map using a plurality of independent learning algorithms and one or more emergent pathogen parameter definition files, wherein:
each of said independent learning algorithms is taught and compiled using a profile corresponding to one or more known pathogens;
at least a portion of said independent learning algorithms independently provide:
a known pathogen parameter output of said target pathogen based on said one or more known pathogen parameters; and
an emergent pathogen parameter output of said target pathogen based on said one or more emergent pathogen parameter definition files, wherein said emergent pathogen parameter output occurs without recompiling any of said learning algorithms;
wherein said emergent pathogen definition files allow said independent learning algorithms to provide said emergent pathogen output without recompiling said learning algorithms or altering said known pathogen parameter outputs; and
combining said known pathogen parameter outputs and emergent pathogen parameter outputs for at least a portion of said independent learning algorithms to make a pathogen determination, thereby

32.-46. (canceled)

47. A pathogen characterization device comprising:
an imaging device for capturing an intensity profile map from a microarray that has been exposed to a material potentially containing a pathogen;
an analyzer having a plurality of independent learning algorithms and emergent pathogen profile definition files, wherein
a) each of said independent learning algorithms is taught and compiled using a profile corresponding to one or more known pathogens;
b) at least a portion of said independent learning algorithms independently provide:
a known pathogen parameter output of said target pathogen based on said one or more known pathogen parameters; and
an emergent pathogen parameter output of said target pathogen based on said one or more emergent pathogen parameter definition files, wherein said emergent pathogen parameter output occurs without recompiling any of said learning algorithms; and
c) wherein said emergent pathogen definition files allow said independent learning algorithms to provide said emergent pathogen parameter output without recompiling said learning algorithms or altering said know pathogen parameter outputs;
wherein said analyzer combines said known pathogen parameter outputs and emergent pathogen parameter outputs for at least a portion of said independent learning algorithms to make a determination, thereby characterizing said pathogen.
